(19) United States
(12) Patent Application Publication
(10) Pub. No.: US 2026/0069623 A1
(43) Pub. Date: Mar. 12, 2026

(54) CORDYCEPIN, DERIVATIVES, COMPOSITIONS AND METHODS THEREOF

(71) Applicant: THE UNIVERSITY OF HONG KONG, HONG KONG (CN)

(72) Inventors: Jiangang SHEN, HONG KONG (CN); Qiaohui DU, HONG KONG (CN)

(73) Assignee: THE UNIVERSITY OF HONG KONG, HONG KONG (CN)

(21) Appl. No.: 19/322,770

(22) Filed: Sep. 9, 2025

Related U.S. Application Data
(60) Provisional application No. 63/692,972, filed on Sep. 10, 2024.

(51) Int. Cl. A61K 31/7076; A61K 31/506; A61P 43/00

(52) U.S. Cl. CPC A61K 31/7076; A61K 31/506; A61P 43/00

(57) ABSTRACT

The present disclosure is in the field of biomedicine and particularly relates to a therapeutic agent for the treatment of mitochondrial disorder and promoting mitochondrial function in a subject. The method comprises administering nucleoside derivative cordycepin CO1, its derivatives and a pharmaceutically acceptable carrier to a subject. Also provided is cordycepin, its derivatives and a pharmaceutical composition thereof.

### RELATED APPLICATIONS

The present application claims priority to U.S. Provisional Application Ser. No. 63/692,972, filed Sep. 10, 2024, which is incorporated by reference in its entirety.

### 1. FIELD

The present disclosure is in the field of biomedicine and particularly relates to a therapeutic agent for the treatment of mitochondrial disorder and promoting mitochondrial function in a subject. The method comprises administering nucleoside derivative cordycepin CO1, its derivatives and a pharmaceutically acceptable carrier to a subject. Also provided is cordycepin, its derivatives and a pharmaceutical composition thereof.

### 2. BACKGROUND

Mitochondrial disorders arise from abnormalities in mitochondrial DNA or nuclear-encoded proteins and enzymes essential for oxidative phosphorylation. Current treatments for mitochondrial diseases remain inadequate, with most strategies limited to supportive measures—such as surgical intervention for ptosis or supplementation with coenzyme Q10 and vitamin E treatment¹.

Mitochondria serve not only as the powerhouse of eukaryotic cells but also play an essential role in determining stem cell fate. Key mitochondrial outputs—including ATP, reactive oxygen species (ROS), and metabolites from the tricarboxylic acid (TCA) cycle—govern processes of self-renewal and differentiation. Recent research indicates that physiological levels of ROS can promote stem cell differentiation^{2′3}. Furthermore, TCA cycle intermediates such as α-ketoglutarate and acetyl-CoA can regulate stem cell fate decisions by modulating epigenetic mechanisms, including DNA and histone demethylation and acetylation^{4′5}.

Mitochondrial diseases result from genetic mutations, either in mitochondrial DNA (mtDNA) or nuclear DNA (nDNA), that affect proteins or RNA molecules essential for mitochondrial function. A puzzling clinical feature of these disorders is their tissue-specific manifestation, where only certain organs are affected despite the ubiquitous presence of mitochondria. This selective vulnerability cannot be fully explained by developmental factors alone and remains poorly understood. Even the tissue-specific expression patterns of certain mitochondrial proteins fail to account for the highly variable spectrum of afflicted organ systems observed in mitochondrial disease syndromes.

Cordycepin, a primary bioactive constituent, is sourced from Cordyceps sinensis, a rare and valuable traditional Chinese medicine. To develop viable alternatives to Cordyceps sinensis, it is critical to identify its active components. Research has shown that Cordyceps sinensis exhibits numerous bioactivities, including anti-inflammatory, anti-fatigue, and anti-tumor effects⁶. Consequently, substitutes such as Paecilomyces hepiali and its extracts are now widely used in preparations. Cordycepin (3′-deoxyadenosine) is a main active ingredient of Cordyceps sinensis, which belongs to a nucleoside analogue, and has excellent effects on the change of cell adaptivity (anti-aging and anti-cancer) during metabolism in the body, immune regulation and inflammation elimination.

Mitochondrial disease stems from functional failure of the mitochondria, which are specialized organelles present in all nucleated human cells. These organelles are responsible for producing over 90% of the cellular energy (ATP) required to sustain life, growth, and function. A loss of mitochondrial efficacy leads to diminished energy production, resulting in cellular damage and potentially cell death. As this process propagates across multiple tissues, entire organ systems can begin to fail, severely compromising the patient's health and lifespan. While mitochondrial disorders predominantly affect children, adult-onset forms are increasingly recognized.

The import of nuclear-encoded proteins into mitochondria is a critical process facilitated by an N-terminal targeting sequence. This sequence directs proteins to receptors on the outer mitochondrial membrane, which oversee their import, processing, and chaperone-assisted assembly into the correct compartment. Although defects in this import system are rare, mutations in associated genes, such as those causing Mohr-Tranebjaerg syndrome⁷, can lead to disease.

Leigh Syndrome (LS) is a severe neurological disorder characterized by bilateral, symmetrical necrotic lesions in subcortical brain regions. It is frequently associated with a systemic deficiency in cytochrome c oxidase (COX, or complex IV). Mutations in SURF1, a nuclear-encoded gene critical for the assembly of COX, are a major cause of Leigh Syndrome. The discovery could provide a potential therapeutic agent to treat Leigh Syndrome.

### 3. SUMMARY

Provided herein is a cordycepin and cordycepin-based derived compound with a potent therapeutic effect for mitochondrial disorders, stem cell therapy and post-stroke therapy. Also provided are compositions comprising cordycepin and cordycepin-based derived compounds. In one embodiment, structure of the compound is shown in FIG. 1.

Provided are methods of preventing and treating mitochondrial disease and disorder. Cordycepin derivatives and a pharmaceutical composition comprising the cordycepin or its derivatives thereof are also provided.

Provided is a method for preventing and treating a disease or disorder by administering cordycepin (“CO1”), its derivative and a pharmaceutically acceptable carrier, to a subject in need thereof, wherein the disease or disorder is a mitochondrial disorder.

In certain embodiments, the cordycepin (“CO1”), its derivative and a pharmaceutically acceptable carrier is a dosage form comprising a tablet, capsule, powder, nanometer powder, solution, suspension, or drip.

In certain embodiments, the derivative comprises cordycepin salt, ester or glycoside.

In certain embodiments, the cordycepin (“CO1”) or its derivative is at a dosage range of 0.5 mg to 1000 mg/kg body weight/day.

In certain embodiments, the subject is a human.

Provided is a pharmaceutical composition comprising cordycepin (“CO1”), its derivative; and a pharmaceutically acceptable carrier, diluent or excipient.

Provided is a composition comprising CO1, its derivative and a suitable GSK3β inhibitor for co-administration for the treatment of a mitochondria disease or disorder.

Provided is a method for alleviating symptoms associated with a mitochondrial disease or disorder, comprising administration of the composition to a subject in need thereof.

Provided is a method of treating or preventing mitochondrial disease or disorder by administering to a subject a compound that inhibit the activity of glycogen synthase kinase 3 beta (GSK3β).

Provided is a method for treating, preventing or alleviating symptoms associated with a mitochondrial disease or disorder, comprising administration of the composition in combination with a second therapeutic agent to a subject in need thereof.

Leigh Syndrome (LS) is a severe neurological disorder characterized by bilateral, symmetrical necrotic lesions in subcortical brain regions. It is frequently associated with a systemic deficiency in cytochrome c oxidase (COX, or complex IV). Mutations in SURF1, a nuclear-encoded gene critical for the assembly of COX, are a major cause of Leigh Syndrome. In certain embodiments, the disclosed method is used to treat Leigh Syndrome or related diseases.

In certain embodiments, the symptoms include one or more of muscle weakness, exercise intolerance, chronic fatigue, gastrointestinal dysmotility, impaired balance, peripheral neuropathy, metabolic strokes, dysautonomia, vision loss, eye muscle and eyelid weakness, hearing loss, glomerular or tubular renal disease, endocrine dysfunction, dyslipidemia, cardiomyopathy, arrhythmia, anemia, failure to thrive, over or underweight, developmental delay, neurodevelopmental regression, cognitive decline and memory impairment, Parkinsonism, dystonia, liver dysfunction or failure, infertility, metabolic instability, stressor-induced acute decompensation DLD disease, Mitophagy disorders, Mitochondrial lipid biogenesis disorders, mitochondrial cofactor disorders, and secondary mitochondrial disorders including but not limited to resulting from toxins, drugs, age, prescribed or illicit medications, smoking, alcohol, environmental exposures, obesity, and genetic disorders that secondarily impair mitochondrial function, structure, or activities.

In certain embodiments, mitochondrial disease or disorder is selected from the group consisting of Complex I disease, Complex II disease, Complex III disease, Complex IV disease, Complex V disease, Multiple respiratory chain complex disease, adenine nucleotide translocase deficiency, pyruvate dehydrogenase deficiency, mitochondrial depletion disease, multiple mitochondrial DNA deletions disease, mitochondrial DNA maintenance defects, mitochondrial translation defects, mitochondrial nucleotide import disease, Friedreich's ataxia, Leber's Hereditary Optic Neuropathy, Kearns-Sayre Syndrome, Pearson Syndrome, Mitochondrial Myopathy, Mitochondrial Encephalomyopathy with Lactic Acidosis and Stroke-Like Episodes, Myoclonic epilepsy and ragged red fibers, Neurogenic Ataxia and Retinitis Pigmentosa, Mitochondrial Neuro-gastrointestinal encephalomopathy, maternally inherited diabetes and deafness, primary lactic acidosis, Leigh syndrome, Leigh-like syndrome, and multi-system mitochondrial disease.

Provided is a method to promote mitochondrial function in mitochondrial dysfunction cells and embryonic stem cells (“ESCs”), said method comprises administering cordycepin CO1, its derivative and a pharmaceutically acceptable carrier, to a subject in need thereof.

### 4. BRIEF DESCRIPTION OF THE FIGURES

FIG. 1. Chemical structure of Cordycepin (CO1).

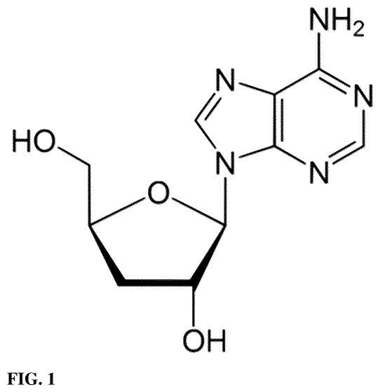

FIG. 2. CO1 increased ATP production in mitochondrial dysfunction patient fibroblasts. Two SURF1 mutated, one SCO2 mutated, one COQ4 mutated and one NDUFA9 mutated fibroblasts were applied to test the effects of CO1 on mitochondrial function. CO1 treatment at dosages of 5, 10 and 20 μM for 48 hours and control group (CTR) were treated in normal culture medium (n=3). Data are presented as mean±S.E.M (*p<0.05, **p<0.01, ***p<0.001, relative to control group, one-way ANOVA).

FIG. 3. CO1 up-regulated mitochondrial complex expression levels in two SURF1 mutated fibroblasts. CO1 treatment at dosages of 5, 10 and 20 μM for 48 hours and control group (CTR) were treated with vehicle in normal culture medium.

FIGS. 4A-C. CO1 enhanced mitochondrial complexes activities in 59 SURF1 mutated fibroblasts. Mitochondrial complex activities were detected by using isolated mitochondria from fibroblasts. The activities were presented as the percentage compared with the normal cells. CO1 treatment at dosages of 5, 10 and 20 μM for 48 hours and control group (CTR) were treated with vehicle in normal culture medium. A. Activity of mitochondrial complex III in CO1 treated 59 SURF1 mutated fibroblast. B. Activity of mitochondrial complex IV in CO1 treated 59 SURF1 mutated fibroblast. C. Activity of mitochondrial complex V in CO1 treated 59 SURF1 mutated fibroblast. Data are presented as mean±S.E.M (*p<0.05, **p<0.01, ***p<0.001, relative to control group, one-way ANOVA).

FIGS. 5A-C. CO1 improved oxygen consumption rate (OCR) in 59 SURF1 mutated fibroblasts. OCR was normalized with protein concentration. 2 μM Oligomycin (Oligo, complex V inhibitor), 4 μM FCCP (a protonophore), 1 μM antimycin A (AntiA, complex III inhibitor) and 1 μM rotenone (Re, complex I inhibitor) were used in the mitochondrial respiration detection. CO1 treatment at dosages of 5, 10 and 20 μM for 48 hours and control group (CTR) were treated with normal medium (n=3). A. Representative OCR in CO1 treated 59 SURF1 mutated fibroblast. B. Quantification of basal mitochondrial OCR in CO1 treated 59 SURF1 mutated fibroblast. C. Quantification of maximum mitochondrial OCR in CO1 treated 59 SURF1 mutated fibroblast. Data are presented as mean±S.E.M (*p<0.05, **p<0.01, ***p<0.001, relative to control group, one-way ANOVA).

FIGS. 6A-C. CO1 interacted with GSK3β and inhibited phosphorylation of GSK3β in ESCs. CO1 directly binding to GSK3β to inhibit phosphorylation of GSK3β. A. Diagram of CO1 binding to NHS-activated magnetic beads. B. Co-Immunoprecipitation results of CO1 in ESCs. C. The expression level of phosphorylation of GSK3β in CO1 treated ESCs.

FIGS. 7A-D. CO1 directly binding to GSK3β to promote mitochondrial functions in surf1 knockout ESCs. A. Surf1 expression in Surf1 knockout ESCs. B. Representative expression levels of GSK3β-PGC1α pathway after CO1 treatment in Surf1 KO ESCs. C. The ratio of mitochondrial DNA to nuclear DNA in Surf1 KO ESCs. D. Thermal stability changes of GSK3β after CO1 incubation in Surf1 KO ESCs.

### 5. DETAILED DESCRIPTION

Provided herein is a novel and potent therapeutic agent for the treatment of mitochondrial disorders. In accordance with the present disclosure, cordycepin (also referred to as CO1) is provided as an active ingredient for use in mitochondrial disorder therapy. The disclosure establishes a novel application of cordycepin in the regulation of mitochondrial function.

Mitochondrial disorders arise from abnormalities in mitochondrial DNA or nuclear-encoded proteins and enzymes essential for oxidative phosphorylation. Current treatments for mitochondrial diseases remain inadequate, with most strategies limited to supportive measures—such as surgical intervention for ptosis or supplementation with coenzyme Q10 and vitamin E.

In one aspect, the nucleoside derivative cordycepin improves mitochondrial function in both fibroblast and embryonic stem cell (ESC) models carrying a surf1 knockout mutation (surf1⁻/⁻).

In certain embodiments, (1) Cordycepin enhances ATP production and increases oxygen consumption rates in surf1⁻/⁻ fibroblasts and surf⁻/⁻ ESCs; (2) Cordycepin improves the activity and expression levels of mitochondrial complexes in fibroblasts; (3) Cordycepin increases mitochondrial membrane potential and boosts ATP production in fibroblasts; and (4) Cordycepin promotes the differentiation potential of surf1⁻/⁻ ESCs into neural stem cells (NSCs).

In one embodiment, provided herein is the compound, cordycepin (also referred to as CO1) as an active ingredient for use in mitochondrial disorder therapy.

In one embodiment, provided is a pharmaceutical composition comprising CO1, its derivatives, and a pharmaceutically acceptable carrier.

In one embodiment, the disclosure establishes a novel application of cordycepin in the regulation of mitochondrial function.

Provided is an application of CO1 or a nucleoside analogue a pharmaceutical composition thereof for preventing and treating a disease or disorder related to a mitochondrial disfunction in a subject.

Compounds as described herein may be administered to the subject. As used herein, a “subject” may be a human, non-human primate, rat, mouse, cow, horse, pig, sheep, goat, dog, cat, etc.

The term “Treatment” is an intervention performed with the intention of altering the pathology or symptoms of a disorder. Those in need of treatment include those already with the disorder.

The term “prevention” in the present disclosure means that the compounds or preparations described herein are administered to prevent one or more symptoms related to the disease or disorder and comprises: preventing a disease or a disease state from appearing in the subject, especially when the subjects are prone to mitochondrial disease and disorder. The subjects that are prone to mitochondrial disease and disorder can be identified by genetic testing well known in the art.

The term “pharmaceutically acceptable” in the present invention aims at those compounds, materials, compositions and/or dosage forms, which are within the range of reliable medical judgment and are suitable for contact with human and animal tissues without excessive toxicity, irritation, allergic reaction or other problems or complications, thus being commensurate with a reasonable benefit/risk ratio.

As used herein, “ameliorated” or “treatment” refers to a symptom which is approaches a normalized value (for example a value obtained in a healthy patient or individual), e.g., is less than 50% different from a normalized value, less than about 25% different from a normalized value, less than 10% different from a normalized value, is not significantly different from a normalized value as determined using routine statistical tests.

### 5.1 Cordycepin

In one embodiment, provided herein is the nucleoside derivative cordycepin, a composition comprising the nucleoside derivative cordycepin, having natural pharmacological activity of the cordycepin. Disclosed herein is a cordycepin derivative, or a pharmaceutically acceptable salt, a stereoisomer, a tautomer, a solvate, a prodrug, or a metabolite thereof.

The compound as described herein may be in the free form or in the form of a salt thereof. In some embodiments, compounds as described herein may be in the form of a pharmaceutically acceptable salt, which are known in the art. Pharmaceutically acceptable salt as used herein includes, for example, salts that have the desired pharmacological activity of the parent compound (salts which retain the biological effectiveness and/or properties of the parent compound and which are not biologically and/or otherwise undesirable).

The compound described herein having one or more functional groups capable of forming a salt may be, for example, formed as a pharmaceutically acceptable salt. Compounds containing one or more basic functional groups may be capable of forming a pharmaceutically acceptable salt with, for example, a pharmaceutically acceptable organic or inorganic acid. Pharmaceutically acceptable salts may be derived from, for example, and without limitation, acetic acid, adipic acid, alginic acid, aspartic acid, ascorbic acid, benzoic acid, benzenesulfonic acid, butyric acid, cinnamic acid, citric acid, camphoric acid, camphorsulfonic acid, cyclopentanepropionic acid, diethylacetic acid, digluconic acid, dodecylsulfonic acid, ethanesulfonic acid, formic acid, fumaric acid, glucoheptanoic acid, gluconic acid, glycerophosphoric acid, glycolic acid, hemisulfonic acid, heptanoic acid, hexanoic acid, hydrochloric acid, hydrobromic acid, hydriodic acid, 2-hydroxyethanesulfonic acid, isonicotinic acid, lactic acid, malic acid, maleic acid, malonic acid, mandelic acid, methanesulfonic acid, 2-napthalenesulfonic acid, naphthalenedisulphonic acid, p-toluenesulfonic acid, nicotinic acid, nitric acid, oxalic acid, pamoic acid, pectinic acid, 3-phenylpropionic acid, phosphoric acid, picric acid, pimelic acid, pivalic acid, propionic acid, pyruvic acid, salicylic acid, succinic acid, sulfuric acid, sulfamic acid, tartaric acid, thiocyanic acid or undecanoic acid.

Compounds containing one or more acidic functional groups may be capable of forming pharmaceutically acceptable salts with a pharmaceutically acceptable base, for example, and without limitation, inorganic bases based on alkaline metals or alkaline earth metals or organic bases such as primary amine compounds, secondary amine compounds, tertiary amine compounds, quaternary amine compounds, substituted amines, naturally occurring substituted amines, cyclic amines or basic ion-exchange resins. Pharmaceutically acceptable salts may be derived from, for example, and without limitation, a hydroxide, carbonate, or bicarbonate of a pharmaceutically acceptable metal cation such as ammonium, sodium, potassium, lithium, calcium, magnesium, iron, zinc, copper, manganese or aluminum, ammonia, benzathine, meglumine, methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, glucamine, methylglucamine, theobromine, purines, piperazine, piperidine, procaine, N-ethylpiperidine, theobromine, tetramethylammonium compounds, tetraethylammonium compounds, pyridine, N,N-dimethylaniline, N-methylpiperidine, morpholine, N-methylmorpholine, N-ethylmorpholine, dicyclohexylamine, dibenzylamine, N,N-dibenzylphenethylamine, i-ephenamine, N,N′-dibenzylethylenediamine or polyamine resins.

In some embodiments, compounds as described herein may contain both acidic and basic groups and may be in the form of inner salts or zwitterions, for example, and without limitation, betaines. Salts as described herein may be prepared by conventional processes known to a person skilled in the arts, for example, and without limitation, by reacting the free form with an organic acid or inorganic acid or base, or by anion exchange or cation exchange from other salts. Those skilled in the art will appreciate that preparation of salts may occur in situ during isolation and purification of the compounds or preparation of salts may occur by separately reacting an isolated and purified compound.

In some embodiments, compounds and all different forms thereof (e.g. free forms, salts, polymorphs, hydrates, hydrated salts, optical isomers, racemates, diastereoisomers, enantiomers, isomeric forms) as described herein may be in a solvent addition form, for example, solvates. Solvates contain either stoichiometric or non-stoichiometric amounts of a solvent in physical association the compound or salt thereof. The solvent may be, for example, and without limitation, a pharmaceutically acceptable solvent. For example, hydrates are formed when the solvent is water or alcoholates are formed when the solvent is an alcohol.

In some embodiments, compounds and all different forms thereof (e.g. free forms, salts, solvates, isomeric forms) as described herein may include crystalline and amorphous forms, for example, polymorphs, pseudopolymorphs, conformational polymorphs, amorphous forms, or a combination thereof. Polymorphs include different crystal packing arrangements of the same elemental composition of a compound. Polymorphs usually have different X-ray diffraction patterns, infrared spectra, melting points, density, hardness, crystal shape, optical and electrical properties, stability and/or solubility. Those skilled in the art will appreciate that various factors including recrystallization solvent, rate of crystallization and storage temperature may cause a single crystal form to dominate.

In some embodiments, the compounds that are useful in the present disclosure may exist in specific geometric or stereoisomeric forms. All such compounds are proposed in the present invention, comprising cis and trans isomers, (−)- and (+)-enantiomers, (R)- and (S)-enantiomers, diastereomers, (D)-isomers, (L)-isomers, racemic mixtures thereof and other mixtures, such as mixtures enriched with the enantiomers or the diastereomers, and all these mixtures are within the scope of the present disclosure. All these isomers and mixtures thereof are included in the scope of the present disclosure.

In some embodiments, compounds and all different forms thereof (e.g. free forms, salts, solvates, polymorphs) as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formulas illustrated for the sake of convenience.

In some embodiments, pharmaceutical compositions as described herein may comprise a salt of such a compound, such as a pharmaceutically or physiologically acceptable salt. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents (used interchangeably herein) are those known in the art for use in such modes of administration.

### 5.2 GSK3 Inhibitors

GSK3β inhibitors are useful in the present disclosure. Specificity for GSK3β may be used to refer to GSK3 inhibitors that specifically or selectively inhibit GSK3β, relative to other glycogen synthase kinases, such as GSK3a. For example, a GSK3 inhibitor that specifically inhibits GSK3β may have an IC₅₀ (μM) that is lower than an IC₅₀ for another glycogen synthase kinase, such as GSK3α. A GSK3 inhibitor that specifically inhibits GSK3β over another glycogen synthase kinase, such as GSK3α, has an IC₅₀ (μM) for GSK3β that is at least 2-times lower, at least 3-times lower, at least 5-times lower, at least 10-times lower, at least 20-times lower, at least 50-times lower, at least 100-times lower, at least 500-times lower, or at least 1000-times lower, than the IC₅₀ (PM) for the other glycogen synthase kinase, such as GSK3α. In some embodiments, the GSK3 inhibitor has an IC₅₀ for GSK3β of less than about 0.050 μM, 0.040 μM, 0.030 μM, 0.020 μM, or 0.010 μM. In some embodiments, the GSK3 inhibitor has an IC₅₀ for GSK3α of greater than about 0.5 μM, 1 μM, 2 μM, 5 μM, or 10 μM. In some embodiments, a daily dose of the GSK3 inhibitors may contain from about 0.01 mg/kg to about 100 mg/kg (such as from about 0.05 mg/kg to about 50 mg/kg and/or from about 0.1 mg/kg to about 25 mg/kg) of the GSK3 inhibitor used in the present method of treatment. The dose may be administered under any suitable regimen (e.g., weekly, daily, twice daily).

### 5.3 Combination Therapy

Instead of administering a pharmaceutical composition comprising a compound, the disclosed methods may be practiced by administering a first pharmaceutical composition and administering a second pharmaceutical composition, where the first composition may be administered before, concurrently with, or after the second composition.

### 5.4 Formulations

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water-soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein micro-particles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20th ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene polyoxypropylene copolymers may be used to control the release of the compounds. Other potentially useful parenteral delivery systems for modulatory compounds include ethylene vinyl acetate copolymer particles, osmotic pumps, implantable infusion systems, and liposomes. Formulations for inhalation may contain excipients, for example, lactose, or may be aqueous solutions containing, for example, polyoxyethylene 9 lauryl ether, glycocholate and deoxycholate, or may be oily solutions for administration in the form of nasal drops, or as a gel.

### 5.5 Route of Administration

The medicaments employed in the present invention can be administered by oral or parenteral routes, including intravenous, intramuscular, intraperitoneal, subcutaneous, transdermal, airway (aerosol), rectal, vaginal and topical (including buccal and sublingual) administration.

For oral administration, the compounds of the invention will generally be provided in the form of tablets, capsules, powder or granules, or as an aqueous solution or suspension.

Tablets for oral use may include the active ingredient mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while cornstarch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredient is mixed with water or an oil such as peanut oil, liquid paraffin or olive oil.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

Compounds or pharmaceutical compositions as described herein or for use as described herein may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

### 5.6 Dosage

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds as described herein should be used without causing substantial toxicity. Toxicity of the compounds as described herein can be determined using standard techniques, for example, by testing in cell cultures or experimental animals and determining the therapeutic index, i.e., the ratio between the LD₅₀ (the dose lethal to 50% of the population) and the LD₁₀₀ (the dose lethal to 100% of the population). In some circumstances however, such as in severe disease conditions, it may be appropriate to administer substantial excesses of the compositions. Some compounds as described herein may be toxic at some concentrations. Titration studies may be used to determine toxic and non-toxic concentrations. Animal studies may be used to provide an indication if the compound has any effects on other tissues.

Useful doses can often range from 0.1 to 5 μg/kg. In other examples, a dose can include 1 μg/kg, 10 μg/kg, 50 μg/kg, 75 μg/kg, 100 μg/kg, 150 μg/kg, 200 μg/kg, 500 μg/kg, 1000 μg/kg, 0.1 to 5 mg/kg, or from 0.5 to 1 mg/kg. In other examples, a dose can include 1 mg/kg, 10 mg/kg, 25 mg/kg, 50 mg/kg, 55 mg/kg, 100 mg/kg, 250 mg/kg, 500 mg/kg, 750 mg/kg, 1000 mg/kg, or more. In particular embodiments, a useful dose is 200 mg/kg/day.

The desired dose may be presented and administered as a single daily dose or as two, three, four, five or six or more sub-doses administered at appropriate intervals throughout the day. Doses may be administered in unit dosage forms, for example, containing 10 to 1500 mg, 20 to 1000 mg, and 50 to 700 mg of active ingredient per unit dosage form. The total daily dose is 1000 to 3000 mg, whether taken as a single dose or as sub-doses at intervals throughout the day.

Each of the described doses of active ingredients can be an active ingredient alone, or in combination with one or more other active ingredients. In particular embodiments, when included in combinations to produce a dose, such as a dose stated herein, the substituents in the combination can be provided in exemplary ratios such as: 1:1; 1:1.25; 1:1.5; 1:1.75; 1:8; 1:1.2; 1:1.25; 1:1.3; 1:1.35; 1:1.4; 1:1.5; 1:1.75; 1:2; 1:3; 1:4; 1:5; 1:6; 1:7; 1:8; 1:9; 1:10; 1:15; 1:20; 1:30; 1:40; 1:50; 1:60; 1:70; 1:80; 1:90; 1:100; 1:200; 1:300; 1:400; 1:500; 1:600; 1:700; 1:800; 1:900; 1:1000; 1:1:1; 1:2:1; 1:3:1; 1:4:1; 1:5:1; 1:10:1; 1:2:2; 1:2:3; 1:3:4; 1:4:2; 1:5:3; 1:10:20; 1:2:1:2; 1:4:1:3; 1:100:1:1000; 1:25:30:10; 1:4:16:3; 1:1000:5:15; 1:2:3:10; 1:5:15:45; 1:50:90:135; 1:1.5:1.8:2.3; 1:10:100:1000 or additional beneficial ratios depending on the number and identity of substituents in a combination to reach the stated dosage. The substituents in a combination can be provided within the same composition or within different compositions.

Therapeutically effective amounts can be achieved by administering single or multiple doses during the course of a treatment regimen (e.g., QID, TID, BID, daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months, or yearly).

### 5.7 Mitochondrial Related Disorders

Mitochondrial related disorders relate to disorders which are due to abnormal mitochondria such as for example, a mitochondrial genetic mutation, enzyme pathways etc. Examples of disorders include and are not limited to: loss of motor control, muscle weakness and pain, gastro-intestinal disorders and swallowing difficulties, poor growth, cardiac disease, liver disease, diabetes, respiratory complications, seizures, visual/hearing problems, lactic acidosis, developmental delays and susceptibility to infection. The mitochondrial abnormalities give rise to “mitochondrial diseases” which include, but not limited to: AD: Alzheimer's Disease; ADPD: Alzheimer's Disease and Parkinsons's Disease; AMDF: Ataxia, Myoclonus and Deafness CIPO: Chronic Intestinal Pseudoobstruction with myopathy and Opthalmoplegia; CPEO: Chronic Progressive External Opthalmoplegia; DEAF: Maternally inherited DEAFness or aminoglycoside-induced Deafnness; DEMCHO: Dementia and Chorea; DMDF: Diabetes Mellitus & Deafness; Exercise Intolerance; ESOC: Epilepsy, Strokes, Optic atrophy, & Cognitive decline; FBSN: Familial Bilateral Striatal Necrosis; FICP: Fatal Infantile Cardiomyopathy Plus, a MELAS-associated cardiomyopathy; GER: Gastrointestinal Reflux; KSS Kearns Sayre Syndrome LDYT: Leber's hereditary optic neuropathy and Dystonia; LHON: Leber Hereditary Optic Neuropathy; LIMM: Lethal Infantile Mitochondrial Myopathy; MDM: Myopathy and Diabetes Mellitus; MELAS: Mitochondrial Encephalomyopathy, Lactic Acidosis, and Stroke-like episodes; MEPR: Myoclonic Epilepsy and Psychomotor Regression; MERME: MERRF/MELAS overlap disease; MERRF: Myoclonic Epilepsy and Ragged Red Muscle Fibers; MHCM: Maternally Inherited Hypertrophic CardioMyopathy; MICM: Maternally Inherited Cardiomyopathy; MILS: Maternally Inherited Leigh Syndrome; Mitochondrial Encephalocardiomyopathy; Mitochondrial Encephalomyopathy; MM: Mitochondrial Myopathy; MMC: Maternal Myopathy and Cardiomyopathy; Multisystem Mitochondrial Disorder (myopathy, encephalopathy, blindness, hearing loss, peripheral neuropathy); NARP: Neurogenic muscle weakness, Ataxia, and Retinitis Pigmentosa; alternate phenotype at this locus is reported as Leigh Disease; NIDDM: Non-Insulin Dependent Diabetes Mellitus; PEM: Progressive Encephalopathy; PME: Progressive Myoclonus Epilepsy; RTT: Rett Syndrome; SIDS: Sudden Infant Death Syndrome.

In one aspect, a method for alleviating symptoms associated with mitochondrial disease is provided. An exemplary method comprises administration of cordycepin or its derivatives, to a subject in need thereof. In certain embodiments, the symptoms include one or more of muscle weakness, exercise intolerance, chronic fatigue, gastrointestinal dysmotility, impaired balance, peripheral neuropathy, metabolic strokes, dysautonomia, vision loss, eye muscle and eyelid weakness, hearing loss, glomerular or tubular renal disease, endocrine dysfunction, dyslipidemia, cardiomyopathy, arrhythmia, anemia, failure to thrive, over or underweight, developmental delay, neurodevelopmental regression, cognitive decline and memory impairment, Parkinsonism, dystonia, liver dysfunction or failure, infertility, metabolic instability, stressor-induced acute decompensation, DLD disease, Mitophagy disorders, Mitochondrial lipid biogenesis disorders, mitochondrial cofactor disorders, and secondary mitochondrial disorders including but not limited to resulting from toxins, drugs, age, prescribed or illicit medications, smoking, alcohol, environmental exposures, obesity, and other primary genetic disorders that secondarily impair mitochondrial function, structure, or activities.

The compositions and methods of the disclosure can be used to treat a mitochondrial disease selected from the group consisting of Complex I disease, Complex II disease, Complex III disease, Complex IV disease, Complex V disease, Multiple respiratory chain complex disease, adenine nucleotide translocase deficiency, pyruvate dehydrogenase deficiency, mitochondrial depletion disease, multiple mitochondrial DNA deletions disease, mitochondrial DNA maintenance defects, mitochondrial translation defects, mitochondrial nucleotide import disease, Friedreich's ataxia, Leber's Hereditary Optic Neuropathy, Kearns-Sayre Syndrome, Pearson Syndrome, Mitochondrial Myopathy, Mitochondrial Encephalomyopathy with Lactic Acidosis and Stroke-Like Episodes, Myoclonic epilepsy and ragged red fibers, Neurogenic Ataxia and Retinitis Pigmentosa, Mitochondrial Neuro-gastrointestinal encephalomopathy, maternally inherited diabetes and deafness, primary lactic acidosis, Leigh syndrome, Leigh-like syndrome, and multi-system mitochondrial disease.

### 5.7.1 Leigh Syndrome

Leigh Syndrome, also known as subacute necrotizing encephalopathy, is a serious disease characterized by psychomotor retardation, seizures, hypotonia and weakness, ataxia, eye abnormalities including vision loss, difficulty in swallowing, and lactic acidosis. The disease can result in lesions to or degeneration of the basal ganglia, thalamus, brain stem, and spinal cord. A disease termed “Leigh-like Syndrome” is also recognized, which is characterized by neurologic abnormalities atypical for but suggestive of Leigh Syndrome. Leigh Syndrome is the most common mitochondrial disease of infancy.

Patients with Leigh Syndrome typically die before the age of five years, often from respiratory failure. Some patients with less severe disease may live to six or seven years, or even into their teen or adult years. Current treatments include thiamine (Vitamin B1), Coenzyme Q, or L-carnitine and oral sodium bicarbonate or sodium citrate to manage lactic acidosis.

The disclosure encompasses a preclinical method for identifying mitochondrial disease subjects likely to respond to treatment for mitochondrial dysfunction. On exemplary method comprises provide patient cell lines or cells from said subject, said subject having a predetermined genotype; contacting said cells with at least one agent; culturing said cells under normal and stressed growth conditions, wherein said stressor is applied in increasing concentrations; and determining the protective effects of said agent on said cells, agents having protective action being effective in subjects having said predetermined genotype. In certain embodiments, tolerability and efficacy of said agent are assessed in a whole animal model of mitochondrial dysfunction. In some embodiments, the cells are contacted with said agent for a time period of between one hour, 2 hours, 5 hours, 1 day, 2 days, 3 days, 4 days, up to 7 days. In some embodiments, the cells are contacted with the stressor for a time period of between one hour, 2 hours, 3 hours, 4 hours, 1 day, 2 days, 3 days, up to four days. Protective effects determined include one or more of improvement in cell viability, cell proliferation, ATP production, mitochondrial membrane protection mitochondrial mass, mitochondrial content, total cellular oxidant levels, cellular pH and oxygen capacity consumption.

### 5.7.2 Mitochondrial Function

Mitochondrial function can be assessed through various methods known by one skilled in the art, including biochemical test, organic acid test, and imaging techniques that evaluate mitochondrial morphology and performance. These assays help assess energy production efficiency, identify potential dysfunction and guide treatment strategies for various metabolic and neurological disorders. For example, promoted mitochondrial function can be measured by (i) an increase in mitochondrial DNA to nuclear DNA ratio; and/or (ii) an increase in expression of a mitochondrial biogenesis regulator. Understanding mitochondrial health is crucial for optimizing cellular bioenergetics and overall health.

### 6. EXAMPLES

### 6.1. CO1 Enhances Mitochondrial Function in Patient-Derived Fibroblasts In Vitro

We investigated the potential of CO1 to improve mitochondrial function using fibroblast cell lines derived from the patients with mutations in various mitochondrial complex subunits. The panel included two cell lines with SURF1mutations (a complex IV assembly factor), and one cell line each with mutations in SCO2 (complex IV), COQ4 (involved in coenzyme Q10 biosynthesis, affecting complexes I, II, and III), and NDUFA9 (complex I). These cell lines were designated by patient number and mutation: 59 surf1⁻/⁻, 96 surf1⁻/⁻, 262 SCO2⁻/⁻, 238 COQ4⁻/⁻, and 191 NDUFA9⁻/⁻.

Fibroblasts were treated with CO1 at concentrations of 5, 10, and 20 μM for 48 hours. We subsequently assessed ATP production, oxygen consumption rate (OCR), and the expression and activity of mitochondrial complexes.

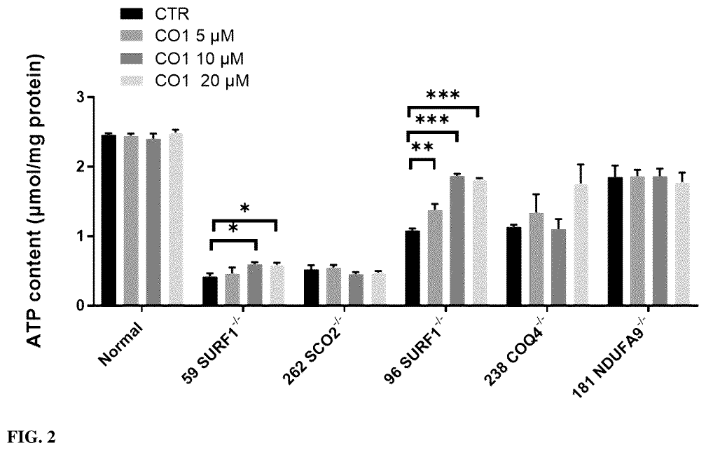

As shown in FIG. 2, CO1 treatment significantly increased ATP production in both SURF1-deficient fibroblast lines. In contrast, the effects of CO1 on healthy control fibroblasts and the other mutation-specific cell lines were minimal.

We next analyzed the expression and enzymatic activity of mitochondrial complexes.

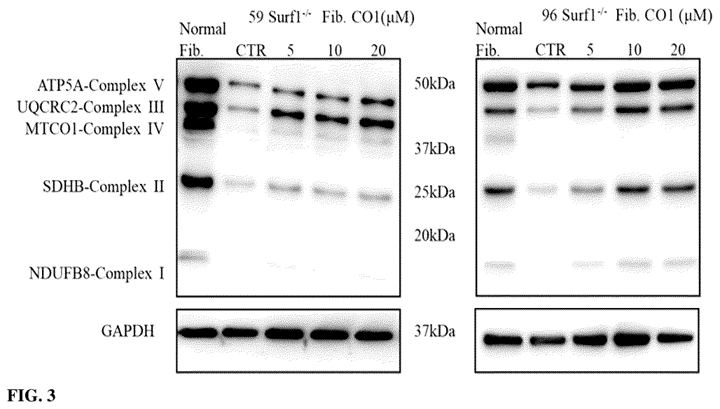

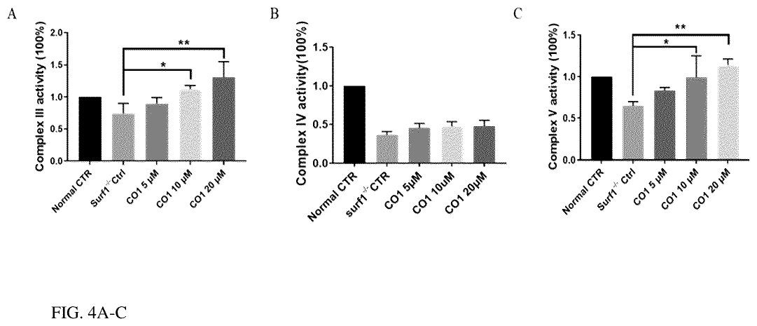

Consistent with the genetic lesion, the expression of complex IV was dramatically reduced in surf1⁻/⁻ fibroblasts compared to controls (FIG. 3). CO1 treatment dose-dependently upregulated the expression of complex III and complex V (ATP synthase) in these cells. Following mitochondrial isolation, we used an ELISA-based activity assay to evaluate the specific enzymatic function of each complex in surf1⁻/⁻ fibroblasts (FIG. 4). The results demonstrated that CO1 remarkably enhanced the activity of complex III and complex V in a dose-dependent manner. In contrast, CO1 had no significant effect on the residual complex IV activity.

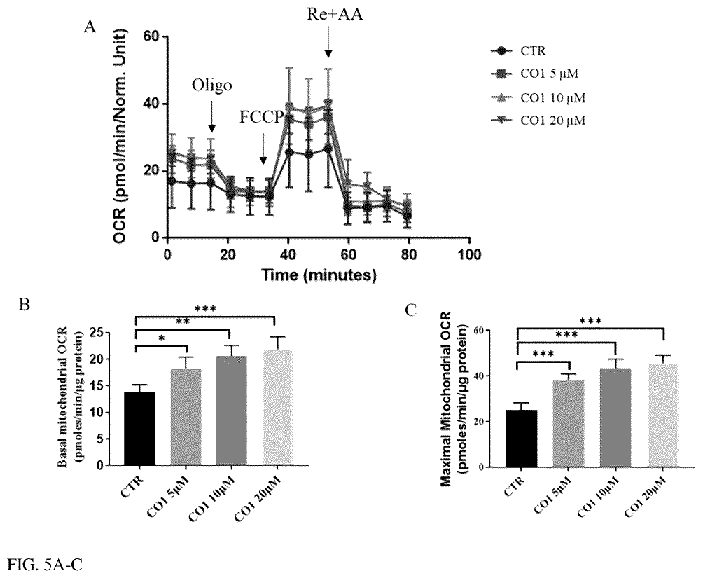

To evaluate overall mitochondrial respiratory function, we measured the oxygen consumption rate (OCR) in CO1-treated surf1⁻/⁻ fibroblasts. Consistent with the above findings, CO1 significantly improved the OCR (FIG. 5), enhancing both basal and maximum mitochondrial respiration capacity.

### 6.2. CO1 Binds to GSK3β and Inhibits its Activity to Promote Mitochondrial Function

To elucidate the mechanism by which CO1 enhances mitochondrial function, we employed a pull-down assay to identify its direct protein targets. NHS-activated magnetic beads were conjugated with CO1, and the resulting CO1-linked beads were incubated with cell lysates to capture binding partners. Potential targets were subsequently identified using liquid chromatography-mass spectrometry (LC-MS).

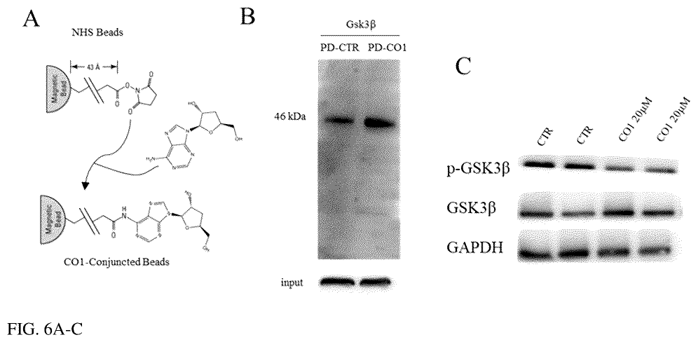

This screen revealed that CO1 directly binds to glycogen synthase kinase 3 beta (GSK3β). This interaction was confirmed in embryonic stem cells (ESCs), where CO1 treatment inhibited the phosphorylation of GSK3β at its active site (FIG. 6), indicating functional inhibition of its kinase activity.

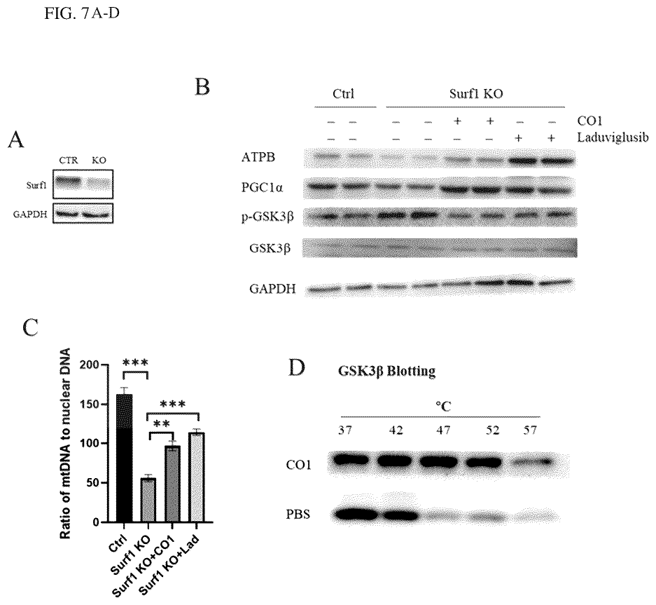

To validate this target in a disease-relevant model, we generated surf1 knockout (KO) ESCs (FIG. 7A). We further employed thermal proteome profiling (TPP), a powerful technique for profiling ligand-protein interactions in their native state. TPP analysis demonstrated that the thermal stability of GSK3β increased upon incubation with CO1 (FIG. 7D), providing strong biophysical evidence for a direct binding interaction.

Functionally, CO1 treatment increased the mitochondrial DNA to nuclear DNA ratio in Surf1 KO ESCs (FIG. 7B), indicating that it stimulates mitochondrial biogenesis. To establish a functional link between GSK3β inhibition and the observed phenotypic effects, we compared CO1 to the established GSK3β inhibitor, Laduviglusib. Both CO1 and Laduviglusib suppressed GSK3β phosphorylation and subsequently promoted the expression of the key mitochondrial biogenesis regulator PGC-1α (FIG. 7C). This result positions GSK3β inhibition as the mechanistic upstream event leading to the activation of pro-mitochondrial pathways.

### 6.3 Materials and Methods

### 6.3.1. Embryonic Stem Cell Culture

The human embryonic stem cell (hESC) lines H7 and H9 were obtained from WiCell Research Institute. hESCs were maintained on 6-well plates coated with growth factor-reduced Matrigel (Corning). Matrigel was thawed overnight at 4° C., diluted 1:30 in DMEM/F12 medium, and 1 mL was added to each well. Coated plates were incubated at 37° C. for at least 1 hour prior to cell seeding. Upon seeding, hESCs were cultured under feeder-free conditions in TeSR-E8 medium (STEMCELL Technologies) supplemented with a ROCK inhibitor (RevitaCell, Gibco).

### 6.3.2. Transmission Electron Microscopy (TEM)

Following 14 days of neural stem cell (NSC) induction with CO1 treatment, NSCs were detached using Accutase (Gibco). Cells were fixed in 4% formaldehyde for 24 hours at 4° C. The fixed cells were then processed through post-fixation in 2.5% glutaraldehyde for 72 hours and 1% osmium tetroxide for 24 hours at 4° C. Subsequently, samples were dehydrated through a graded ethanol series, permeabilized with propylene oxide, and embedded in EPON resin. Polymerized blocks were sectioned into 90 nm ultrathin slices, which were stained with 3% uranyl acetate and 1% lead citrate. Digital images were acquired using a Phillips CM100 transmission electron microscope.

### 6.3.3. Mitochondrial Isolation

Mitochondria were isolated from fibroblasts using a commercial mitochondrial isolation kit (Abcam). Briefly, cells were dissociated with trypsin, resuspended in Buffer A, and incubated on ice for 10 minutes. The cell suspension was then homogenized using a glass homogenizer. The homogenate was centrifuged to remove nuclei and cell debris, and the resulting supernatant was further centrifuged at 12,000×*g* for 10 minutes to pellet the mitochondria. The mitochondrial pellet was resuspended in Buffer C for subsequent activity assays.

### 6.3.4. Enzyme-Linked Immunosorbent Assay (ELISA)

The activities of mitochondrial complexes III, IV, and V were assessed using ELISA kits (Abcam). Isolated mitochondria were added to a microplate pre-blocked with BCA and incubated for 4 hours. Detection was performed according to the manufacturer's instructions. Absorbance was measured kinetically every minute for 60 minutes at room temperature using a multi-plate reader (Bio-Rad Model 680). Complex III and IV activities were read at 550 nm, and complex V activity was read at 340 nm.

### 6.3.5. Cell Cycle Analysis

After two days of induction and CO1 treatment, ESCs were labeled with 20 μM BrdU for 1 hour. Cells were then detached using Accutase, permeabilized with 0.1% Triton X-100 for 10 minutes, and stained with a FITC-conjugated anti-BrdU antibody and 7-AAD to label incorporated BrdU and total DNA, respectively. After staining, cells were washed with PBS and analyzed by flow cytometry.

### 6.3.6. Targeted Metabolomics

Cell extracts were prepared identically to the ATP/ADP/AMP assay. Metabolite analysis was performed using an Agilent 7890B GC system coupled to an Agilent 7010 Triple Quadrupole Mass Spectrometer. Separation was achieved on a DB-5MS capillary column (30 m×0.25 mm ID, 0.25 μm) with helium as the carrier gas at a constant flow of 1 mL/min. The oven temperature program was: 60° C. (hold 1 min), ramp at 10° C./min to 120° C., then 3° C./min to 150° C., then 10° C./min to 200° C., and finally 30° C./min to 280° C. (hold 5 min). The inlet and transfer line temperatures were set to 250° C. and 280° C., respectively. Data were acquired in both SCAN (m/z 50-500) and MRM modes. Analytes were quantified using Agilent MassHunter Quantitative Analysis software with external standard calibration curves.

### 6.3.7. Neural Progenitor Cells Differentiation

C17.2 neural progenitor cells were subjected to oxygen-glucose deprivation (OGD) to induce injury. For OGD, cells were cultured in glucose-free DMEM and placed in a hypoxia chamber (Eppendorf New Brunswick Galaxy 48R) perfused with a gas mixture of 0.1% O₂, 5% CO₂, and balance N₂ for 2 hours. After OGD, cells were returned to normal culture medium and placed in a normoxic incubator (5% CO₂/95% air) for 24 hours of reoxygenation. Subsequently, cells were incubated in differentiation medium for 14 days. The differentiation medium consisted of a 1:1 mixture of two media: (1) DMEM/F12 supplemented with 1% N2, 1% non-essential amino acids, and 1% GlutaMAX; and (2) Neurobasal medium supplemented with 2% B27, 10 ng/mL BDNF, and 10 ng/mL GDNF. To investigate the role of mitochondrial function and the Akt pathway, cells were treated with BYC extract (200 μg/mL) in the presence or absence of oligomycin (Oligo, 1 μM) or wortmannin (Wort, 0.5 μM).

### 6.3.8. Embryonic Stem Cells Differentiation

Human embryonic stem cells were cultured in TesR-E8 medium in Matrigel pre-coated 6 well plate. After 24 hours of recovery from ROCK inhibitor, the cells were incubated with differentiation medium for 14 days. The differentiation medium was consisted of two separate media: (1) DMEM/F12 (Gibco) supplemented with 1% N2 (Gibco), 1% non-essential amino acid (Gibco) and 1% GlutaMAX (Gibco); and (2) neurobasal (Gibco) supplemented with 2% B27 (Gibco), 10 μM SB431542 and 5 μM LDN193189 (Goparaju et al., 2017). After 7 days differentiation, LDN193189 inhibitor was removed from day 7 to day 14.

### 6.3.9. Oxygen Consumption Rate (OCR)

The Seahorse XF24 Extracellular Flux Analyzer (Seahorse Bioscience, North Billerica, MA) was applied to test mitochondrial function in ESCs and fibroblasts. Prior to the start of the experiment, ESCs were seeded (7,000 cells/well) into the XF24 cell culture plate and allowed to attach for 24 hours in E8 medium. After 48 hours CO1 treatment in differentiation medium, the culture medium was replaced with XF medium (Seahorse Bioscience). Cells were placed in a non-CO2 37° C. incubator for 1 hour, prior to start of the experiment. OCR and extracellular acidification rate (ECAR) were detected over a 3-minute period, followed by 3 min mixing and re-oxygenation of the medium. Following the establishment of a basal OCR reading, oligomycin (1 μM) is used to inhibit proton (H+) flow through blocking ATP synthase. Maximal respiration can be initiated by exposing cells to Carbonyl cyanide-ptrifluoromethoxyphenyl hydrazone (FCCP, 0.5 μM), which is an ionophore that transports H+ across the mitochondrial membrane. Antimycin A (1 μM) and rotenone (1 μM) prevents mitochondrial respiration by blocking complex I and III. Three measurements of OCR/ECAR were obtained following injection of each drug. OCR and ECAR readings were normalized to total protein levels (BCA protein assay, Pierce) in each well. Each dosage was represented in 3 wells per experiment and replicate experiments carried out at least three times.

### 6.3.10. Immunofluorescence

The collected brain tissue was post-fixed with 4% paraformaldehyde (PFA) for 48 hours, completely dehydrated in 30% sucrose solution at 4° C. and embedded in O.C.T. The entire length of the dentate gyrus was cut into 30 m sections as frozen slices and stored at −20° C. For NSCs imaging, the cells were cultured in Poly-D-Lysine coated 12 mm microscope slides (GmbH & Co. KG, Germany). Brain samples were processed with antigen-retrieved citrate acid buffer (pH 6.0) and microwave for 30 min. The samples were permeabilized and blocked with PBS containing 5% goat serum and 0.3% Triton X-100 for 1 hour at room temperature. For BrdU labelling detection, sections were incubated with 2N HCl for 30 min at 37° C. and rinsed in 0.1 M borate buffer (pH 8.5) before blocking. For cell slides, the samples were fixed with 4% PFA at room temperature for 20 min and blocked with 5% goat serum. After blocking, the samples were incubated with primary antibodies and stained with fluorochrome conjugated secondary antibodies, counterstained the nucleus with DAPI and mounted with antifade medium (Dako). Cell images were obtained by regular confocal microscope (Zeiss LSM 800, Germany; Core facility in LSK Faculty of Medicine, HKU) and analyzed by Zeiss software.

### 6.3.11. Western Blotting

Proteins samples from brain tissue and cells were extracted by radioimmunoprecipitation assay buffer plus with 1% protease and phosphatase inhibitor cocktails (Cell Signaling Technology). Protein concentration was tested by BCA Protein Assay Kit (Thermo Fisher Scientific), separated by sodium dodecyl sulfate-polyacrylamide (SDS-PAGE) gel electrophoresis and transferred on 0.45 μM polyvinylidene fluoride (PVDF) membranes. The membrane was blocked by 5% bovine serum albumin solution for 1 hour at room temperature and immunoblotted with primary antibodies following HRP-conjugated secondary antibodies, including GSK-30, (Cell signaling, 9315), Phospho-GSK-3-beta (Ser9) (Cell signaling 9322), mitochondrial complex antibody (Abcam, ab110413), ATPB (Abcam, ab110280), PGC1α (Abcam, ab191838). Immunoreactive bands on the membrane were revealed by chemiluminescent ECL select kit (GE Healthcare), visualized by Gel-Doc system (Bio-Rad) and analyzed by Image Lab software (Bio-Rad).

### Exemplary Products, Systems and Methods are Set Out in the Following Items:

1. A method for treating a mitochondrial disease or disorder, or for promoting mitochondrial function in a subject in need thereof, the method comprising administering an effective amount of cordycepin (“CO1”) or its derivative and a pharmaceutically acceptable carrier to the subject.
2. The method of item 1 wherein the derivative comprises cordycepin salt, ester or glycoside.
3. The method of any one of the preceding items wherein the mitochondrial disorder is related to a SURF-1 deficiency.
4. The method of any one of the preceding items, wherein the subject has a complete deletion, partial deletion or mutation of a SURF-1 gene.
5. The method of any one of the preceding items wherein the mitochondrial disorder is a cytochrome c oxidase (“COX”) deficiency in the subject.
6. The method of any one of the preceding items wherein the subject has increased basal and mitochondrial respiration capacity after administration of the cordycepin (“CO1”), its derivative and a pharmaceutically acceptable carrier to the subject.
7. The method of any one of the preceding items, wherein the mitochondrial disease or disorder is selected from the group consisting of Complex III disease, Complex V disease, Mitochondrial Encephalomyopathy, Mitochondrial Neuro-gastrointestinal encephalomopathy, Leigh syndrome, Leigh-like syndrome, multi-system mitochondrial disease or a combination thereof.
8. The method of any one of the preceding items wherein the mitochondrial disorder is Leigh syndrome or Leigh-like syndrome.
9. The method of any one of the preceding items wherein the cordycepin (“CO1”) or its derivative is at a dosage range of 0.5 mg/kg body weight/day to 1000 mg/kg body weight/day.
10. The method of any one of the preceding items wherein the cordycepin (“CO1”), its derivative and a pharmaceutically acceptable carrier is administered for 24-48 hours.
11. The method of any one of the preceding items wherein the cordycepin (“CO1”), its derivative and a pharmaceutically acceptable carrier is a dosage form comprising a tablet, capsule, powder, nanoparticle, solution, suspension, or drip.
12. The method of any one of the preceding items wherein the cordycepin (“CO1”), its derivative is at a concentration of 3 μM-5 μM, 5 μM-10 μM, 1 μM-20 μM, 20 μM-30 μM, or 30 μM-50 μM in a composition comprising a pharmaceutically acceptable carrier.
13. The method of any one of the preceding items wherein the subject is a human.
14. The method of any one of the preceding items wherein the CO1 specifically binds to glycogen synthase kinase 38 (“GSK3β”).
15. The method of any one of the preceding items further comprising administration of an effective amount of GSK3β inhibitor.
16. The method of item 15 wherein the GSK3β inhibitor is Laduviglusib.
17. The method of any one of the preceding items wherein the mitochondrial function is promoted in mitochondrial dysfunctional cells in the subject.
18. The method of any one of the preceding items wherein the promotion of mitochondrial function is measured by: (i) an increase in mitochondrial DNA to nuclear DNA ratio; and/or (ii) an increase in expression of a mitochondrial biogenesis regulator.
19. The method of item 18 wherein the mitochondrial biogenesis regulator is peroxisome proliferator-activated receptor 7 coactivator 1α (“PGC-1α”).
20. The method of any one of the preceding items, wherein the mitochondrial disease or disorder has symptoms selected from the group consisting of muscle weakness, exercise intolerance, chronic fatigue, gastrointestinal dysmotility, impaired balance, peripheral neuropathy, metabolic strokes, dysautonomia, vision loss, eye muscle and eyelid weakness, hearing loss, glomerular or tubular renal disease, endocrine dysfunction, dyslipidemia, cardiomyopathy, arrhythmia, anemia, failure to thrive, over or underweight, developmental delay, neurodevelopmental regression, cognitive decline and memory impairment, Parkinsonism, dystonia, liver dysfunction or failure, infertility, metabolic instability, stressor-induced acute decompensation DLD disease, Mitophagy disorders, Mitochondrial lipid biogenesis disorders, mitochondrial cofactor disorders, and a combination thereof.
21. A composition comprising cordycepin (“CO1”), its derivative; and a pharmaceutically acceptable carrier.
22. A method for alleviating symptoms associated with a mitochondrial disease or disorder, comprising administration of the composition of item 21 to a subject in need thereof.
23. The method of any one of the preceding items, wherein the symptoms include one or more of muscle weakness, exercise intolerance, chronic fatigue, gastrointestinal dysmotility, impaired balance, peripheral neuropathy, metabolic strokes, dysautonomia, vision loss, eye muscle and eyelid weakness, hearing loss, glomerular or tubular renal disease, endocrine dysfunction, dyslipidemia, cardiomyopathy, arrhythmia, anemia, failure to thrive, over or underweight, developmental delay, neurodevelopmental regression, cognitive decline and memory impairment, Parkinsonism, dystonia, liver dysfunction or failure, infertility, metabolic instability, stressor-induced acute decompensation DLD disease, Mitophagy disorders, Mitochondrial lipid biogenesis disorders, mitochondrial cofactor disorders, and secondary mitochondrial disorders including but not limited to resulting from toxins, drugs, age, prescribed or illicit medications, smoking, alcohol, environmental exposures, obesity, and genetic disorders that secondarily impair mitochondrial function, structure, or activities.
24. The method of any one of the preceding items, wherein said mitochondrial disease or disorder is selected from the group consisting of Complex I disease, Complex II disease, Complex III disease, Complex IV disease, Complex V disease, Multiple respiratory chain complex disease, adenine nucleotide translocase deficiency, pyruvate dehydrogenase deficiency, mitochondrial depletion disease, multiple mitochondrial DNA deletions disease, mitochondrial DNA maintenance defects, mitochondrial translation defects, mitochondrial nucleotide import disease, Friedreich's ataxia, Leber's Hereditary Optic Neuropathy, Kearns-Sayre Syndrome, Pearson Syndrome, Mitochondrial Myopathy, Mitochondrial Encephalomyopathy with Lactic Acidosis and Stroke-Like Episodes, Myoclonic epilepsy and ragged red fibers, Neurogenic Ataxia and Retinitis Pigmentosa, Mitochondrial Neuro-gastrointestinal encephalomopathy, maternally inherited diabetes and deafness, primary lactic acidosis, Leigh syndrome, Leigh-like syndrome, and multi-system mitochondrial disease.
25. A method to promote mitochondrial function in mitochondrial dysfunction cells and embryonic stem cells (“ESCs”), said method comprises administering cordycepin CO1, its derivative and a pharmaceutically acceptable carrier, to a subject in need thereof.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

1. Hausse, A., Aggoun, Y., Bonnet, D., Sidi, D., Munnich, A., Rotig, A., and Rustin, P. J. H. (2002). Idebenone and reduced cardiac hypertrophy in Friedreich's ataxia. 87, 346-349.
2. Khacho, M., Clark, A., Svoboda, D. S., Azzi, J., MacLaurin, J. G., Meghaizel, C., Sesaki, H., Lagace, D. C., Germain, M., Harper, M. E., et al. (2016). Mitochondrial Dynamics Impacts Stem Cell Identity and Fate Decisions by Regulating a Nuclear Transcriptional Program. Cell Stem Cell 19, 232-247. 10.1016/j.stem.2016.04.015.
3. Lyublinskaya, O., Borisov, Y. G., Pugovkina, N., Smirnova, I., Obidina, J. V., Ivanova, J. S., Zenin, V., Shatrova, A., Borodkina, A., Aksenov, N. J. O.m., and longevity, c. (2015).

Reactive oxygen species are required for human mesenchymal stem cells to initiate proliferation after the quiescence exit. 2015.
4. Kaelin Jr, W. G., and McKnight, S. L. J. C. (2013). Influence of metabolism on epigenetics and disease. 153, 56-69.
5. Wellen, K. E., Hatzivassiliou, G., Sachdeva, U. M., Bui, T. V., Cross, J. R., and Thompson, C. B. J. S. (2009). ATP-citrate lyase links cellular metabolism to histone acetylation. 324, 1076-1080.
6. Yan, J.-K., Wang, W.-Q., and Wu, J.-Y. (2014). Recent advances in Cordyceps sinensis polysaccharides: Mycelial fermentation, isolation, structure, and bioactivities: A review. Journal of Functional Foods 6, 33-47.
7. Tranebjaerg, L., Schwartz, C., Eriksen, H., Andreasson, S., Ponjavic, V., Dahl, A., Stevenson, R. E., May, M., Arena, F., and Barker, D. J. J. o.m.g. (1995). A new X linked recessive deafness syndrome with blindness, dystonia, fractures, and mental deficiency is linked to Xq22. 32, 257-263.
8. Jaimes, R., Kuzmiak-Glancy, S., Brooks, D. M., Swift, L. M., Posnack, N. G., and Kay, M. W. (2016). Functional response of the isolated, perfused normoxic heart to pyruvate dehydrogenase activation by dichloroacetate and pyruvate. Pfluigers Archiv—European Journal of Physiology 468, 131-142. 10.1007/s00424-015-1717-1.
9. Wang, C. H. et al. (2020). The novel application of cordycepin in maintaining stem cell pluripotency and increasing iPS cell generation efficiency. Scientific Reports 10:2187

What is claimed:

1. A method for treating a mitochondrial disease or disorder, or for promoting mitochondrial function in a subject in need thereof, the method comprising administering an effective amount of cordycepin (“CO1”) or its derivative and a pharmaceutically acceptable carrier to the subject.

2. The method of claim 1 wherein the derivative comprises cordycepin salt, ester or glycoside.

3. The method of claim 1 wherein the mitochondrial disorder is related to a SURF-1 deficiency.

4. The method of claim 1, wherein the subject has a complete deletion, partial deletion or mutation of a SURF-1 gene.

5. The method of claim 1 wherein the mitochondrial disorder is a cytochrome c oxidase (“COX”) deficiency in the subject.

6. The method of claim 1 wherein the subject has increased basal and mitochondrial respiration capacity after administration of the cordycepin (“CO1”), its derivative and a pharmaceutically acceptable carrier to the subject.

7. The method of claim 1, wherein the mitochondrial disease or disorder is selected from the group consisting of Complex III disease, Complex V disease, Mitochondrial Encephalomyopathy, Mitochondrial Neuro-gastrointestinal encephalomopathy, Leigh syndrome, Leigh-like syndrome, multi-system mitochondrial disease or a combination thereof.

8. The method of claim 7 wherein the mitochondrial disorder is Leigh syndrome or Leigh-like syndrome.

9. The method of claim 1 wherein the cordycepin (“CO1”) or its derivative is at a dosage range of 0.5 mg/kg body weight/day to 1000 mg/kg body weight/day.

10. The method of claim 1 wherein the cordycepin (“CO1”), its derivative and a pharmaceutically acceptable carrier is administered for 24-48 hours.

11. The method of claim 1 wherein the cordycepin (“CO1”), its derivative and a pharmaceutically acceptable carrier is a dosage form comprising a tablet, capsule, powder, nanoparticle, solution, suspension, or drip.

12. The method of claim 1 wherein the cordycepin (“CO1”), its derivative is at a concentration of 3 μM-5 μM, 5 μM-10 μM, 10 μM-20 μM, 20 μM-30 μM, or 30 μM-50 μM in a composition comprising a pharmaceutically acceptable carrier.

13. The method of claim 1 wherein the subject is a human.

14. The method of claim 1 wherein the CO1 specifically binds to glycogen synthase kinase 3β (“GSK3β”).

15. The method of claim 1 further comprising administration of an effective amount of GSK3β inhibitor.

16. The method of claim 15 wherein the GSK3β inhibitor is Laduviglusib.

17. The method of claim 1 wherein the mitochondrial function is promoted in mitochondrial dysfunctional cells in the subject.

18. The method of claim 1 wherein the promotion of mitochondrial function is measured by: (i) an increase in mitochondrial DNA to nuclear DNA ratio; and/or (ii) an increase in expression of a mitochondrial biogenesis regulator.

19. The method of claim 18 wherein the mitochondrial biogenesis regulator is peroxisome proliferator-activated receptor γ coactivator 1α (“PGC-1α”).

20. The method of claim 1, wherein the mitochondrial disease or disorder has symptoms selected from the group consisting of muscle weakness, exercise intolerance, chronic fatigue, gastrointestinal dysmotility, impaired balance, peripheral neuropathy, metabolic strokes, dysautonomia, vision loss, eye muscle and eyelid weakness, hearing loss, glomerular or tubular renal disease, endocrine dysfunction, dyslipidemia, cardiomyopathy, arrhythmia, anemia, failure to thrive, over or underweight, developmental delay, neurodevelopmental regression, cognitive decline and memory impairment, Parkinsonism, dystonia, liver dysfunction or failure, infertility, metabolic instability, stressor-induced acute decompensation DLD disease, Mitophagy disorders, Mitochondrial lipid biogenesis disorders, mitochondrial cofactor disorders, and a combination thereof.
